(19) United States
(12) Patent Application Publication
(10) Pub. No.: US 2016/0271343 A1
(43) Pub. Date: Sep. 22, 2016

(54) PORTABLE NEBULIZER APPARATUS FOR MEDICAL USE, WITH EASY ACCESS TO THE MESH ASSEMBLY

(71) Applicant: FLAEM NUOVA S.P.A., Desenzano del Garda (Brescia) (IT)

(72) Inventors: Luigi ABATE, Desenzano del Garda (Brescia) (IT); Riccardo ABATE, Desenzano del Garda (Brescia) (IT); Nicola FRACCAROLI, Desenzano del Garda (Brescia) (IT)

(73) Assignee: FLAEM NUOVA S.P.A., Desenzano del Garda (Brescia) (IT)

(21) Appl. No.: 14/442,336

(22) PCT Filed: Nov. 12, 2013
(86) PCT No.: PCT/IB2013/002507

§ 371 Date: May 12, 2015

(30) Foreign Application Priority Data
Nov. 12, 2012 (IT) MI2012A001923

(51) Int. Cl. A61M 11/00; A61M 15/00

(52) U.S. Cl. CPC A61M 11/001; A61M 15/0021; A61M 2205/50; A61M 2205/8206

(57) ABSTRACT

Portable nebulizer apparatus for medical use, comprising a mesh arranged so as to intercept an internal fluid path which leads into a dispenser outlet, and an oscillator element which causes said mesh to oscillate, the mesh being integrated in an extractable mesh assembly housed between a first and a second half-casing, the half-casings being movable in relation to each other between a coupled operational configuration and a separated configuration which allows access to the mesh assembly.

### FIELD OF APPLICATION

The present invention relates to a nebulizer apparatus for medical use, in particular to a nebulizer apparatus of the portable vibrating-mesh type.

The aforementioned nebulizer apparatus is usefully employed in the medical sector, in particular in the sector of aerosol therapy.

### PRIOR ART

The administration of medicinal products aerially by means of special nebulization devices is a therapeutic practice normally used in connection with many illnesses, in particular those affecting the respiratory apparatus.

Nebulization allows in particular a medicinal product in liquid form to be divided up into a plurality of very fine particles, having a diameter of a few micrometres which may be easily inhaled by a patient.

In addition to conventional compressed-air nebulizers, so-called ultrasonic nebulizers have been developed over time, these being lighter and more silent and making use of the high-frequency vibration of a piezoelectric element in order to atomize the liquid medicinal product contained inside a reservoir.

Recently ultrasonic nebulizers have been further improved with the introduction of vibrating mesh technology (VMT). Instead of providing a piezoelectric element on the bottom of the nebulizer reservoir, this new technology envisages the insertion in a dispenser outlet of a very thin membrane or “mesh” provided with a multitude of micrometric holes. By causing the mesh to vibrate at a high frequency, very fine nebulization of the medicinal product through the holes is obtained, without, however, encountering the problems of hyperthermia and excessive dispersion of the product, which constitute the limitations of conventional ultrasonic nebulizers.

Owing to the lightness and small-volume characteristics of VMT technology, portable vibrating-mesh nebulizer apparatuses may be advantageously provided. These devices, which are typically battery-powered, have a reduced weight and sizse so as to be able to be easily transported by the patient and also used outside of the domestic or hospital environment.

While fully meeting the needs of the sector, the portable nebulizers with a vibrating mesh currently present on the market nevertheless have a drawback associated with the need for maintenance of the device.

The models known hitherto, in fact, while offering the possibility of accessing the assembly comprising the mesh element in order to perform cleaning or replacement thereof, require for this purpose relatively long or complex operations, if not even the use of the special tools. The difficult nature of said maintenance is not compatible with the intended aims of a portable device, the main prerogative being that it should be easy and quick to use in any conditions.

The prior art document US 2008/0006264 discloses a nebulizer device which forms the preamble of claim 1.

The technical problem underlying the present invention is therefore that of devising a portable nebulizer apparatus with vibrating mesh which allows easy access to the mesh assembly for cleaning and/or replacement operations, without moreover adversely affecting the robustness and stability of the casing containing the mesh assembly nor resulting in the risk of unintentional opening.

### SUMMARY OF THE INVENTION

The aforementioned technical problem is solved by a portable nebulizer apparatus for medical use, comprising a mesh arranged so as to intercept an internal fluid path which leads into a dispenser outlet, and an oscillator element which causes said mesh to oscillate. The mesh is advantageously integrated in an extractable mesh assembly housed between a first and a second half-casing, said half-casings being movable in relation to each other between a coupled operational configuration and a separated configuration which allows access to said mesh assembly.

The integration of the mesh inside an assembly which can be easily removed fey opening the two half-casings facilitates significantly the operations for maintenance of the nebulizer apparatus; in particular the replacement and/or cleaning of the mesh do not require laborious operations for disassembly of the device.

In particular, the half-casings are hinged together along a first hinging axis.

This measure allows simple and immediate separation of the two half-casings by means of a hinged opening system.

The two aforementioned half-casings define a dispenser part of the nebulizer apparatus, said dispenser part being removably mounted on a handgrip part.

The handgrip part defines a constraint which prevents relative rotation of the two half-casings about the hinging axis when the dispenser part is mounted on top of it; such a measure constitutes a guarantee as to the structural stability of the device in the operating position.

The constraint is preferably defined by the contact between counter-shaped profiles of the handgrip part and the dispenser part, this contact preventing the movement closer together of the two portions of the profile of the dispenser part formed on the first and second half-casing, respectively.

The nebulizer apparatus may also comprise first releasable restraining means arranged at a distance from the first hinging axis so as to lock the two-half-casings in the coupled configuration.

These first releasable restraining means, which preferably comprise a retaining tooth which is associated with the first half-casing and can be snap-engaged inside a through-slot of the second half-casing, facilitate advantageously the stability of the device in its operating configuration.

In an alternative embodiment, said first releasable restraining means may comprise at least one retaining tooth associated with the first half-casing and lockable inside a dedicated blind seat of the second half-easing by means of a dedicated latch which can be operated from the outside of said second half-casing.

In this case, the retaining teeth are preferably two in number and the two latches are formed so as to project from the side walls of the second half-casing, defining a corresponding number of release buttons.

The apparatus may also comprise a reservoir for containing a medicinal product in the liquid state, in communication with the internal fluid path.

This reservoir is preferably integrated in said first half-casing and is accessible at the top by means of a cover so as to be able to be easily topped up by the operator or patient.

The cover may be hinged, along a second hinging axis, at the free end of the second half-easing (i.e. at the end situated opposite to that end already hinged with the first half-casing). The apparatus may comprise second releasable retaining means arranged at a distance from the second hinging axis so as to constrain the cover in a position where it closes the first half-shell.

It can be noted how the configuration proposed above has the advantage of a simple and compact structure which offers the patient the possibility of easy access to the reservoir (by means of rotation of the cover with respect to the second half-casing coupled to the first half-casing) and then to the mesh assembly (by means of further rotation of the second half-casing with respect to the first half-casing).

The second restraining means described above may replace or supplement the first restraining means; they may advantageously comprise a lug articulated with the cover and provided with an engaging tooth designed to snap-lock underneath a projection integral with the first half-casing.

The handgrip part may advantageously incorporate internally the batteries and control electronics of the device and have on its outer surface the interface commands for the operator or patient. The internal fluid path is instead formed in the dispenser part which comprises internally the reservoir, mesh assembly and oscillator element. Electrical contacts are provided between the two portions which connect the control electronics to the oscillator element.

The dispenser part and the handgrip part may be engaged together by means of the attachment means, said attachment means comprising an externally accessible release pushbutton.

In particular, the attachment means may comprise at least one guide which can be coupled with at least one counter-guide so as to provide a sliding constraint between dispenser part and handgrip part, said attachment means further comprising a retaining tooth which is designed to snap-lock together the said parts upon reaching an end-of-travel stop of the sliding constraint and is releasable by pressing the release pushbutton.

The mesh assembly defined above may advantageously comprise an annular support for the mesh, said annular support being shaped so as to provide a form-fit with the internal shapes of the two half-casings.

This measure allows rapid and precise positioning of the mesh inside its seat.

Moreover, in order to facilitate insertion of the mesh assembly, one of the two half-casings (preferably the second half-casing) may comprise a plurality of radial ribs intended to guide insertion of the mesh assembly into its correct position.

The nebulizer apparatus may also comprise a mouthpiece which engages in a known manner on the dispenser outlet. This mouthpiece may advantageously comprise, in addition to a dispenser aperture intended to come into contact with the patient's mouth, at least one lateral membrane intended to limit the dispersion of fluid into the surrounding environment during exhalation of the patient and aspiration of ambient air—which is mixed together with the nebulized medicinal product—during inhalation of the patient.

It should be noted how the lateral dispersion of the fluid during exhalation prevents advantageously any formation of condensate inside the nebulization chamber.

The membranes may be of any number and have any shape or position, provided that the aforementioned advantage is achieved and that it remains within the scope of the present invention.

Further characteristic features and advantages will appear more clearly from the detailed description provided hereinhelow of a preferred, but not exclusive embodiment of the present invention, with reference to the attached figures provided by way of a non-limiting example.

### BRIEF DESCRIPTION OF THE DRAWINGS

FIG. 1 shows a front view of the nebulizer apparatus according to the present invention;

FIG. 2 shows a side view of the nebulizer apparatus according to FIG. 1;

FIG. 3 shows a view, from above, of the nebulizer apparatus according to FIG. 1;

FIGS. 4-11 show successive operations of a method for extracting a mesh assembly from the nebulizer apparatus according to FIG. 1;

FIG. 12 shows a perspective view of the nebulises apparatus according to FIG. 1, comprising an inhalation mouthpiece;

FIG. 13 shows an enlarged detail of the inhalation mouthpiece according to FIG. 12;

FIGS. 14 and 15 show two perspective views of an alternative embodiment of the nebulizer apparatus according to the present invention, shown in the open position with the mesh assembly removed and in position, respectively;

### DETAILED DESCRIPTION

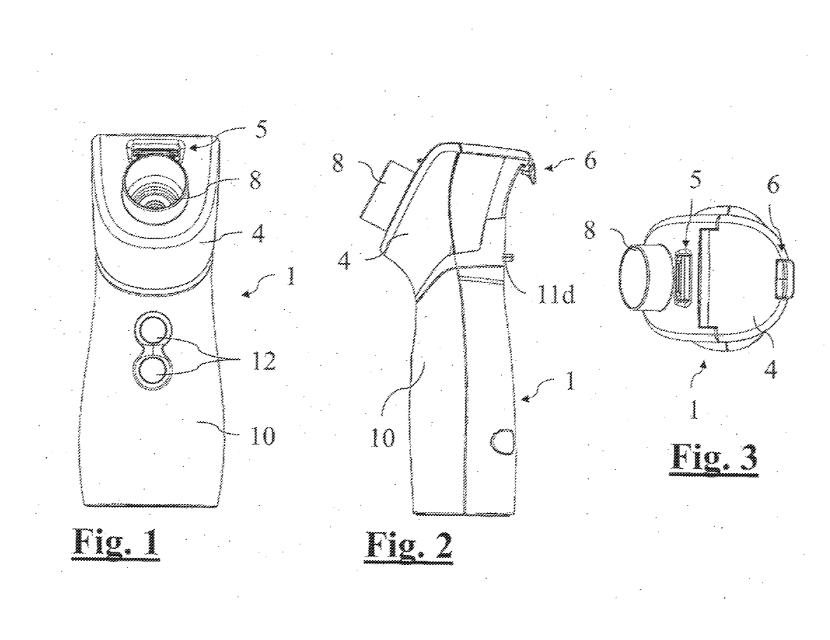

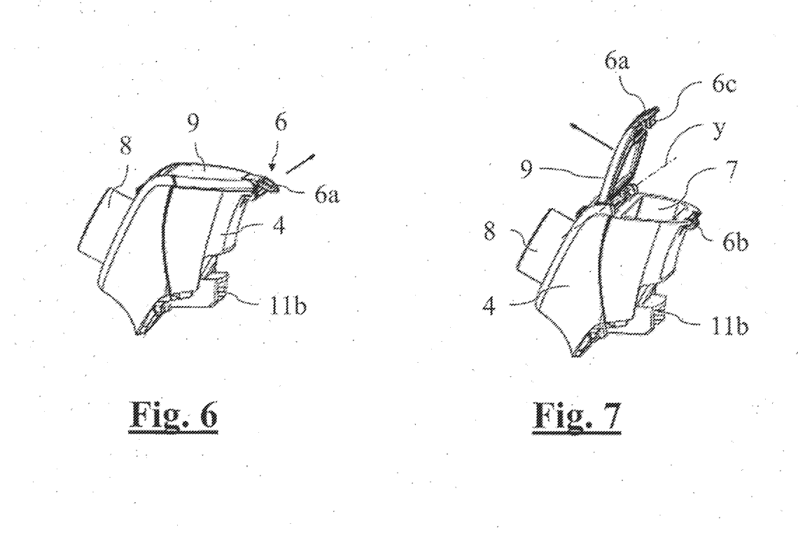

With reference to the attached FIGS. 1-4, 1 denotes in general a nebulizer apparatus for medical use, in particular for aerosol therapy.

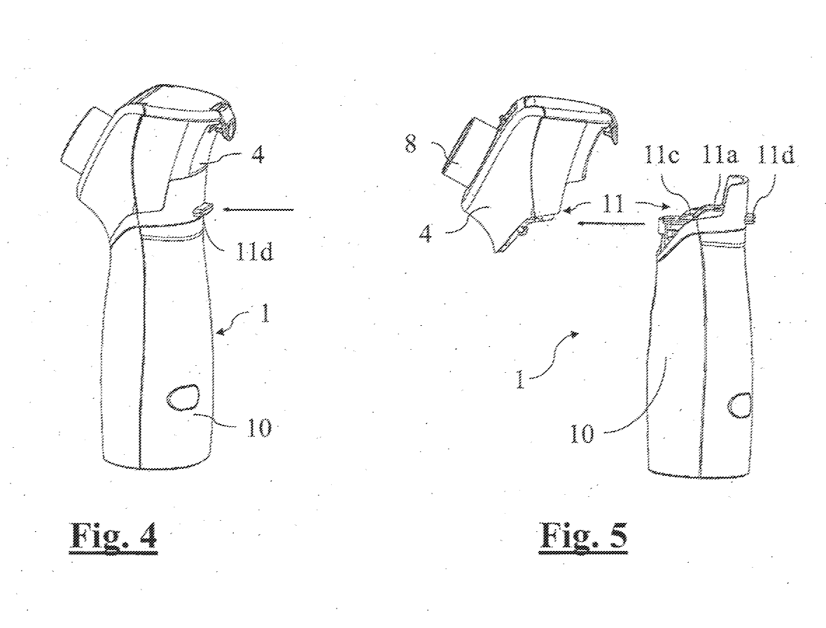

Such a nebulizer apparatus 1 is shown in FIGS. 1 and 2, arranged vertically, in a preferred operating configuration; below in the present description, the positions and orientations, relative and absolute, of the various parts which make up the device and which are defined using terms such as “upper” and “lower”, “above” and “below”, “horizontal” and “vertical” or other equivalent terms, must always be interpreted with reference to this configuration.

The nebulizer apparatus 1, which uses the vibrating mesh technology (VMT), is in particular of the portable type, namely having dimensions and weight such as to allow daily transportation of the device and use in environments other than a domestic and/or hospital environment.

The nebulizer apparatus 1 is composed essentially of two structural parts arranged on top of each other; a handgrip part 10 and a dispenser part 4 arranged on top.

The handgrip part 10, which is formed so as to be able to be easily gripped by the patient during therapy, comprises internally the electronic control components of the device, not visible in the figures. This part further comprises an externally accessible control interface which in the case in question consists of two control pushbuttons 12 situated on a front side of the nebulizer apparatus 1.

The handgrip part 10 may also comprise a space for housing the power supply batteries (not visible in the figures) and/or a connector (not visible in the figures) suitable for connection to an external power supply source, for directly powering the device and/or recharging said batteries.

The dispenser part 4, which is removably mounted above the handgrip part by means of attachment means 11 described in detail below, instead comprises most of the functional components which produce the actual nebulization effect.

This dispenser part 4 comprises in particular a reservoir 7 for containing a medicinal product in the liquid state, which can be accessed from above by means of a dedicated re-closable cover 9. The dispenser part 4 also comprises a dispenser outlet 8 for the nebulized liquid which is situated at the front of the nebulker apparatus 1.

A fluid path, which passes through a mesh 2a arranged at the entrance of the dispenser outlet 8, is defined between the reservoir 7 and dispenser outlet 8. An oscillator element 3 is arranged upstream of said mesh 2a, one end thereof being arranged in contact with said mesh 2a and being designed to cause oscillation thereof. The oscillator element 3 is suitably connected to the control electronics of the handgrip part 10 by means of electrical contacts which connect the latter to the dispenser part 4.

The mesh 2a is in the form of a vibrating foil which has, formed in it, a plurality of micrometric nebulization holes which allow nebulization of a medical liquid to be dispensed; it has a small thickness, typically of less than one tenth of a millimetre.

The mesh 2a is incorporated in a mesh assembly 2 comprising, in addition to the foil mentioned above, an annular support 2b which allows easy-handling of the element and precise assembly thereof inside its seat upstream of the dispenser outlet 8.

In the case in question, the annular support 2b consists of two rings which enclose between them a peripheral seal, for example overmoulded on the mesh 2a. The two rings are shaped so as to match the inner coupling surfaces of the dispenser part 4 with which they mate.

In order to allow easy and immediate extraction of the mesh assembly 2 described above, the dispenser part 4 of the dispenser apparatus comprises a first half-casing 4a and a second half-casing 4b which define respectively the upstream part and downstream part of the fluid path and enclose between them the mesh assembly 2.

The first half-casing 4a and the second half-casing 4b are hinged along a first hinging axis x arranged along their bottom joining point, namely along the interface with the handgrip part 10. The hinge thus allows hinged opening of the two half-casings 4a, 4b for extraction of the mesh assembly 2.

The dispenser part 4 is therefore movable between an operating configuration, in which the two half-casings 4a, 4b are coupled together and define continuously the fluid path, and a maintenance configuration, in which the two half-casings 4a, 4b are separated in order to allow extraction of the mesh assembly 2 arranged between them.

The first half-casing 4a incorporates internally the reservoir 7 and the oscillator element 3; the dispenser outlet 8 of the device is instead formed on the second half-casing 4b.

Only the second half-casing 4b has a hollow structure, which is closed in the operating position against a convex front wall of the reservoir 7 of the first half-casing 4a. The convex front wall has, in its bottom part, a projection provided with a flat surface 13a which has a circular opening 13b in the centre thereof. The circular opening 13b, which communicates with the bottom of the reservoir 7, also houses the vibrating end of the oscillator element 3; when the device is in the operating position, said end is positioned in contact with the mesh 2a.

The mesh assembly 2 is therefore locked between the aforementioned flat surface 13a and an inner end of a cylindrical sleeve 13c which defines externally the outlet mouth 8. The mesh 2a is thus positioned between the aforementioned circular opening 13b and the inlet of the cylindrical sleeve 13c, intercepting the fluid path from the reservoir 7.

Releasable restraining means 5 are provided at the top of the two half-casings 4a, 4b so as to lock together the said half-casings 4b in the coupled configuration. In particular, these releasable restraining means comprise an elastic retaining tooth 5a projecting from the convex front wall of the first half-casing 4a and a corresponding through-slot 5b formed in the second half-casing 4b. In the operating position, the retaining tooth 5a is introduced inside the through-slot 5b, so as to snap-lock inside it. The tip of this retaining tooth 5 is thus accessible from the outside of the device and may be manually raised should it be required to release the restraining means 5 in order to allow opening of the two half-casings 4a, 4b.

The cover 9 of the reservoir 7 has an approximately semicircular form corresponding to an upper shape of the first half-casing 4a. The base of said semicircular cover 9 is hinged, along a second hinging axis y parallel to the first hinging axis x, on the top end of the second half-casing 4b. When the two half-casings 4a, 4b are located in the coupled operating position, the hinge allows opening of the cover 9 with respect to the reservoir 7 of the first half-casing 4a.

Second releasable restraining means 6 are provided, being arranged at a distance from the second hinging axis x or y so as to constrain the cover 9 in the closed position with respect to the first half-casing 4a. In the case in question, these restraining means 6 comprise an articulated lug 6a associated with the end of the cover 9 opposite to the hinged end, said lug 6a being provided with an engaging tooth 6c designed to be snap-engaged underneath a projection 6b integral with a rear surface of the first half-shell 4a.

The dispenser part 4 is coupled on top of the handgrip part 10 by means of the aforementioned attachment means 11.

These attachment means 11 comprise rectilinear guides 11a formed on the top of the handgrip part 10 and able to be coupled with counter-guides 11b integral with the bottom surface of the dispenser part 4. The guides 11a and counter-guides 11b form a sliding constraint which allows horizontal sliding of the dispenser part 4 on top of the handgrip part 10 until an end-of-travel stop defined by the mutual counter-shaped forms of the two parts is reached. A locking tooth 11c, which is arranged underneath the guides 11a of the handgrip part, is raised when said end-of-travel position is reached, resulting in locking together of the two parts 4, 10 in the assembled configuration. A release pushbutton 11d, which is kinematically connected to said locking tooth 11c, is also provided on the rear surface of the handgrip part 10. In response to a pressure applied on the release pushbutton 11d, the kinematic connection results in lowering of the locking tooth 11c and a corresponding release of locking together of the two parts 4, 10 of the nebulizer apparatus 1.

It should be noted that the attachment means 11, with the sole exception of the release pushbutton 11d, are all housed inside the external counter-shaped casings of the two coupled parts 4, 10, such that they are not visible when the device is assembled.

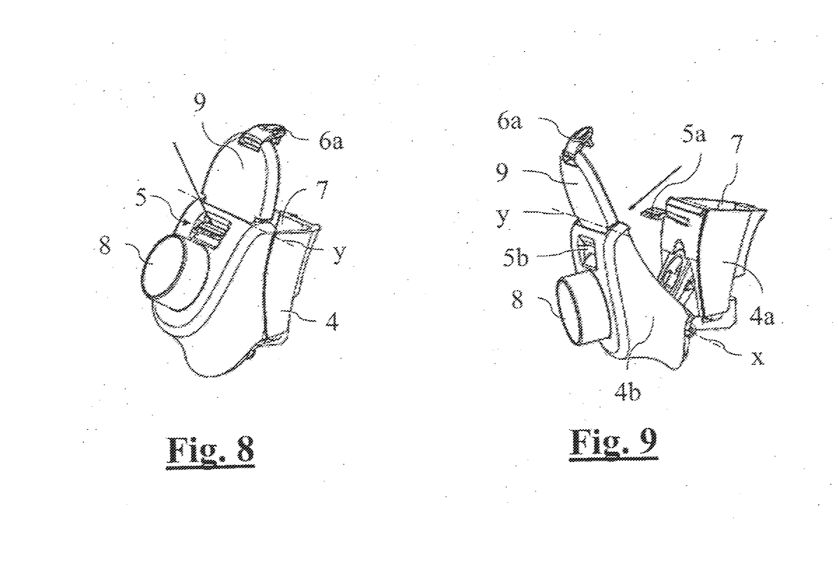

With reference to FIGS. 4-11, a procedure for extracting the mesh assembly 2 from the nebulizer apparatus 1, in order to perform replacement or cleaning thereof, is described below.

First of all, as can be seen from FIGS. 4 and 5, the dispenser part 4 must be separated from the handgrip part 10. In fact, given the counter-shaped form of the contact profiles of the casings of the two parts 4, 10, in the assembled condition the handgrip part acts as a further kinematic, constraint, preventing relative rotation of the two half-casings 4a, 4b about the first hinging axis x.

In order to perform separation of the dispenser part 4, first of all the release pushbutton 11d is operated, causing lowering of the locking tooth 11c and release of the locked together condition of the two parts 4, 10 (FIG. 4). A forwards displacement of the dispenser part 4 with respect to the handgrip part 10 is then performed until the two parts are completely disengaged (FIG. 5).

During a following step the engaging tooth 6c of the hinged lug 6a is disengaged from the projection 6b in order to release the rear end of the cover 9 (FIG. 6). The cover 9 is then raised (FIG. 7).

During a following step the exposed part of the retaining tooth 5a is operated so as to release it from the through-slot 5b (FIG. 8). The two half-casings 4a, 4b are then splayed by performing a relative rotation about the first hinging axis (FIGS. 9 and 10).

Lastly, the mesh assembly 2 which is finally accessible is extracted.

The same steps described above may be repeated in the reverse order in order to restore the operating apparatus 1 to the assembled operational configuration.

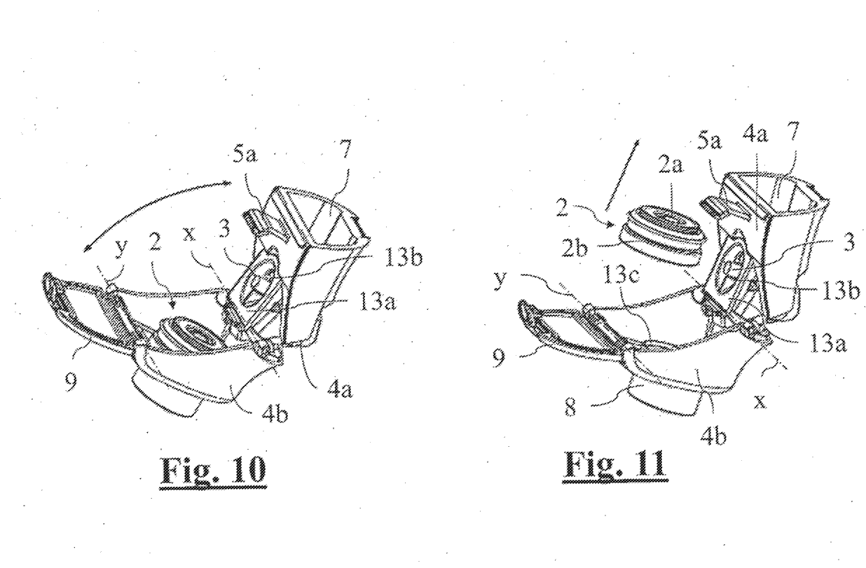

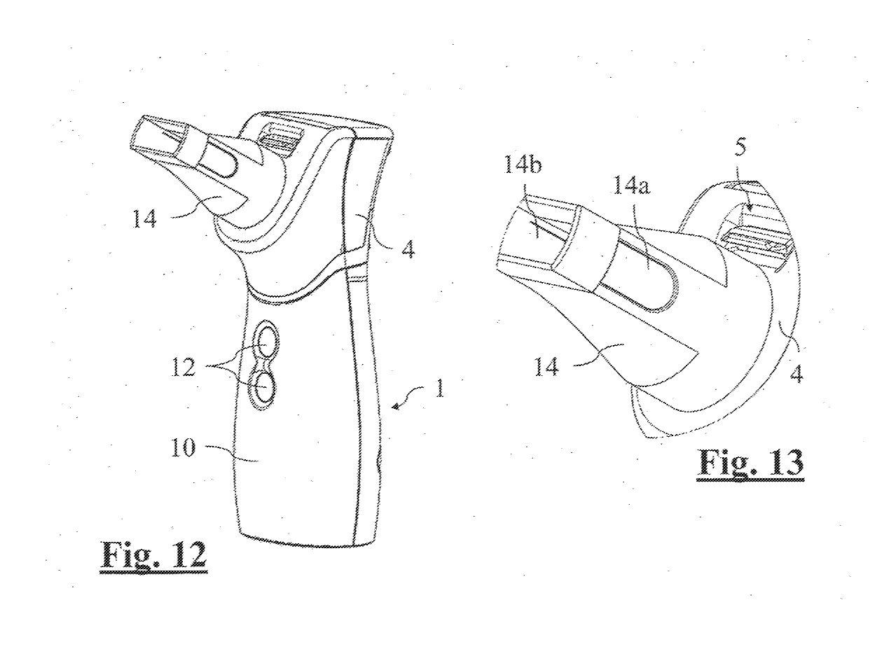

As can be seen in FIGS. 12 and 13, a mouthpiece 14 intended to come into direct contact with the patient's lips can be removably engaged on top of the dispenser outlet 8.

This mouthpiece 14, in the preferred, but not exclusive embodiment described with reference to the figures, has a sleeve portion, intended to be fitted on top of the dispenser outlet, and a following nozzIe portion, which leads into an elongated dispenser aperture 14b intended to be gripped between the patient's lips.

Two lateral membranes 14a, only one of which is visible in the accompanying figures, are provided along the lateral flanks of the nozzle portion. These membranes are gas-permeable. External air is therefore able to access the inside of the mouthpiece 14 during inhalation of the medicinal product; this external air is mixed with the nebulized product and is sucked in by the patient. The same lateral membranes 14a limit the dispersion of the exhalation into the external environment, preventing the formation of condensate inside the nebulization chamber of the device.

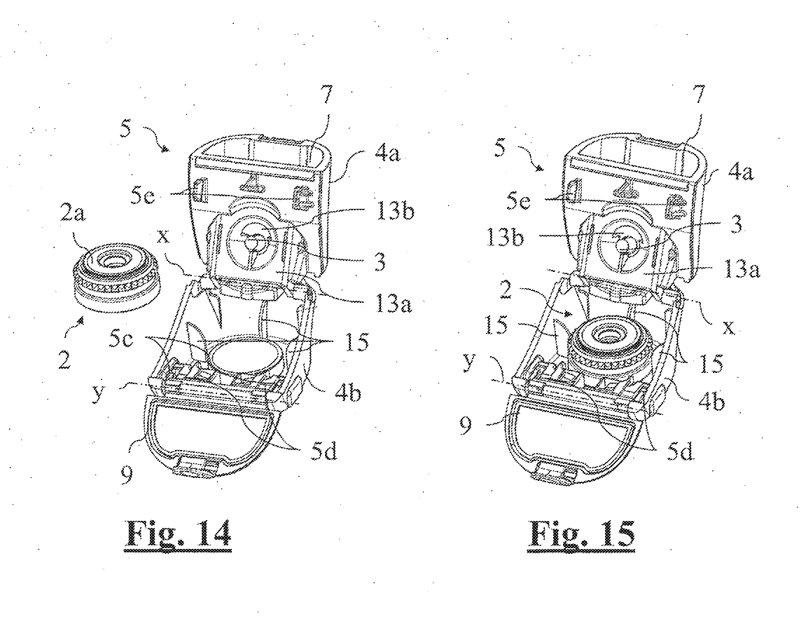

With reference to the attached FIGS. 14 and 15, an alternative embodiment of the dispenser part of the nebulizer apparatus according to the present invention is described below.

It can be seen that said alternative embodiment retains most of the characteristic features of the previously described embodiment. Similar parts have been identified in the figure using the same reference numbers and their function may be understood by referring to the previous description.

It should be noted, moreover, that the dispenser part according to the present embodiment may be mounted on a handgrip part 10 which is entirely similar to that described above.

A first distinguishing characteristic of the alternative embodiment relates to the form of the releasable restraining means 5.

In the case in question, the releasable restraining means comprise two retaining teeth 5e which are associated with the first half-casing 4a and are symmetrically arranged on the two sides of the latter at the end situated furthest away from the first hinging axis x.

These teeth engage inside two blind seats 5c which are formed in the second half-casing 4b. In particular, the two blind seats 5c are defined by the eyelets of corresponding latches 5d which are slidably associated with the inside of the second half-casing 4b and are movable in a direction parallel to that of the hinging axes x and y.

The two latches 5d are subject tc the action of elastic means which in the case in question are formed by an eiastically deformable foot and tend to push the said latches towards the outer side wall of the half-casing. The action of these elastic means locks in position the two retaining teeth 5e when the latter are pushed inside the blind seats 5c so as to produce the snap-engagement of the two half-casings 4a, 4b.

The latches 5d both have a lug projecting laterally outside of the second half-casing 4b, so as to define two release pushbuttons which are externally accessible by the user. The release pushbuttons allow the latches 5d to be pushed inwards, against the action of the aforementioned elastic means, so as to release the retaining teeth 5e which are trapped inside the respective blind seats 5c.

A second distinguishing feature of the alternative embodiment relates to the presence of a plurality of radial ribs 15 provided on the inner surface of the second half-casing 4b and intended to guide insertion of the mesh assembly 2 into its correct operating position.

In the case in question, the radial ribs 15, which are formed as one piece with the second half-casing 4b, are three in number and angled at 90° with respect to each other, the edge adjacent to the cover 9 being the only one not provided with such a rib.

Obviously, a person skilled in the art, in order to satisfy any specific requirements which arise, may make numerous modifications and variations to the invention described above, all of these moreover being contained within the scope of protection of the invention, as defined by the following claims.

1. A portable nebulizer apparatus for medical use, comprising a mesh arranged so as to intercept an internal fluid path which leads into a dispenser outlet and an oscillator element which causes said mesh to oscillate, said mesh being integrated in an extractable mesh assembly housed between a first and a second hall-casing, said half-casings being hinged with each other along a first hinging axis and movable in relation to each other between a coupled operational configuration and a separated configuration which allows access to said mesh assembly; said two half-casings defining a dispenser part of the nebulizer apparatus; wherein said dispenser part is removably mounted on a handgrip part, the handgrip part defining a constraint which prevents the relative rotation of the two half-casings around the hinging axis when said dispenser part is mounted on the handgrip part.

2. The nebulizer apparatus according to claim 1, further comprising first releasable restraining means located at a distance from the first hinging axis so as to lock the two-half-casings in the coupled configuration.

3. The nebulizer apparatus according to claim 2, wherein said first releasable restraining means comprise a retaining tooth associated with the first hall-casing and snap-engageable inside a through-slot on the second half-casing.

4. The nebulizer apparatus according to claim 2, wherein said first releasable restraining means comprise at least one retaining tooth associated with the first half-casing and lockable inside a corresponding blind seat of the second half-casing by means of a dedicated latch which can be operated from the outside of said second half-casing.

5. The nebulizer apparatus according to claim 1, further comprising a reservoir for containing a medicinal product in the liquid state, in communication with the internal fluid path, said reservoir being integrated in said first half-casing and accessible from above by means of a cover.

6. The nebulizer apparatus according to claim 5, wherein said cover is hinged, along a second hinging axis, on the free end of the second half-casing, said nebulizer apparatus further comprising second releasable restraining means located at a distance from the second hinging axis so as to constrain the cover in a position where it closes the first half-casing.

7. The nebulizer apparatus according to claim 1, wherein said dispenser part and said handgrip part can be mutually engaged by attachment means, said attachment means comprising at least one release button which is externally accessible.

8. The nebulizer apparatus according to claim 7, wherein said attachment means comprise at least one guide which can be coupled with at least one counter-guide so as to create a sliding restraint between dispenser part and handgrip part, said attachment means further comprising a locking tooth which is designed to snap-lock together the said parts (4,10) upon reaching an end-of-travel stop of the sliding constraint and is releasable by pressing the release button.

9. The nebulizer apparatus according to claim 1, wherein said mesh assembly comprises an annular support for the mesh shaped so as to provide a form-fit with the internal shapes of the two half-casings.

10. The nebulizer apparatus according to claim 1, wherein one of the two half-casings comprises a plurality of radial ribs intended to guide the insertion of the mesh assembly into its correct position.

11. The nebulizer apparatus according to claim 1, further comprising a mouthpiece fitted onto the dispenser outlet, said mouthpiece comprising a dispenser aperture intended to come into contact with the patient's mouth and at least one lateral membrane intended to limit the dispersion of fluid into the surrounding environment during exhalation of the patient and aspiration of ambient air during inhalation of the patient.
